(19) United States
(12) Patent Application Publication
(10) Pub. No.: US 2013/0259825 A1
(43) Pub. Date: Oct. 3, 2013

(54) Method for Enhancing Umbilical Cord Blood Engraftment

(71) Applicant: William Tse, Beachwood, OH (US)

(72) Inventor: William Tse, Beachwood, OH (US)

(21) Appl. No.: 13/854,451

(22) Filed: Apr. 1, 2013

Related U.S. Application Data
(60) Provisional application No. 61/618,865, filed on Apr. 2, 2012.

(51) Int. Cl. C12N 5/077; C12N 5/078; C12N 5/073

(52) U.S. Cl. CPC C12N 5/0673; C12N 5/0603; C12N 5/0634
USPC 424/85.1; 435/405; 435/325

(57) ABSTRACT

A method for enhancing bone marrow cells, peripheral blood, and umbilical cord blood engraftment is disclosed wherein at least one of a granulocyte colony stimulating factor and a granulocyte macrophage colony stimulating factor, and optionally romiplostim, and optionally an erythropoiesis-stimulating agent, are added thereto for enhancing the engraftment of CD44 cells within the bone marrow cells, peripheral blood, and umbilical cord blood. Also provided is a method for enhancing a stem cell infusion by activating an up-regulation of a AF1q/CD44 signaling pathway. A stem cell product is disclosed having treated stem cells conditioned for activating an up-regulation of a AF1q/CD44 signaling pathway.

### CROSS-REFERENCE TO RELATED APPLICATION

This utility patent application claims the benefit of priority to co-pending U.S. Provisional Patent Application Ser. No. 61/618,865 filed Apr. 2, 2012. The entire contents of U.S. Provisional Patent Application Ser. No. 61/618,865 are incorporated by reference in its entirety into this utility patent application.

### BACKGROUND OF THE INVENTION

1. Field of the Invention

This invention relates to a method for enhancing umbilical cord blood (hereinafter “UCB”) engraftment. Specifically, the present invention method comprises adding to umbilical cord blood an amount of at least one of a granulocyte colony stimulating factor and a granulocyte macrophage colony stimulating factor, and an amount of romiplostim, and an amount of an erythropoiesis-stimulating agent. The erythropoiesis-stimulating agent is preferably epoetin alfa for enhancing engraftment.

2. Background Art

Allogeneic stem cell transplantation is an effective approach to cure many hematopoietic malignant disorders. However, the chance of having full human leukocyte antigen (HLA) matched marrow and blood hematopoietic stem cells in a given patient are somewhere around 30% (from matched sibling donors) to 70% (from unrelated donors). The chance is even lower for non-Caucasian recipients because they usually have very low registration rates in the world-wide hematopoietic stem cell donor program. The requirement of the UCB HLA match is lower compared to conventional stem cell sources requiring a minimal of 4/6 HLA matched between UCB and recipient for transplantation. The chance of getting a minimal matched UCB for transplantation in a given recipient is more than 95%. Umbilical cord blood (UCB) is a valuable alternative source of hematopoietic stem cells for patients that require allogeneic transplantation in the absence of readily available human leukocyte antigen (HLA) matched marrow and blood hematopoietic stem cells. However, there are several biologic hurdles in UCB transplantation that need to be resolved in order to widely expand the UCB application in adult stem cell therapy (SCT): (1) relatively lower hematopoietic stem cell dosage in each UCB unit relative to adult recipients limits the use for adult recipients, and (2) UCB requires longer engraftment time which has been related to higher peri-transplantation morbidity and mortality compared to conventional stem cell sources. In order to overcome the lower cell dosage in UCB units and shorten the engraftment duration, using double units of UCB for adult recipients has been a common practice. However, there is still no reliable methodology to predict and promote the engraftment of a particular UCB unit. Thus, there is a significant unmet need to develop strategies that can help clinicians to effectively identify which UBC unit will ultimately engraft, and more importantly, to promote a biologically desirable UCB unit (e.g. better HLA matched to recipients) engraftment.

Using double UCB units for adult hematopoietic stem cell recipients is a well established strategy to treat their hematopoietic malignant disorders. In order to overcome the lower cell dosage in UCB units as stated hereinabove, it is common to use double units for adult recipients, however, this practice has the potential for increasing the risk of protracted chronic graft-versus-host disease. Thus, if we can effectively identify which UBC unit ultimately engrafts and most importantly promote the biologically desirable UCB unit, such as those UCB units that have relatively lower cell dosage but better HLA match with recipients in the double UCB transplantation setting to engraft, this will significantly improve the UCB transplantation safety and application for adult patients. To date, there is still no reliable methodology to predict and promote the engraftment UCB unit. Using an ex vivo approach to expand the UCB cell dose has met with some successes but has yet to be conclusively tested in the double UCB transplantation setting.

The AF1q gene was identified by the present inventor and co-investigators (see W. Tse et al. “A novel gene, AF1q, fused to MLL in t(1;11)(q21;q23), is specifically expressed in leukemic and immature hematopoietic cell”, Blood, Vol. 85, pages 650-656 (1995)), as a mixed lineage leukemia (MLL) fusion partner in acute myeloid leukemia (AML) patients whose leukemic blasts carrying a t(1; 11) (q2i; q23) translocation. AF1q is expressed in normal hematopoietic precursors including CD34+ cells, but has very little or no expression in mature hematopoietic blood cells.

### SUMMARY OF THE INVENTION

The present invention provides a method for enhancing umbilical cord blood engraftment comprising providing an amount of an umbilical cord blood, adding to the umbilical cord blood an effective amount of at least one of a granulocyte colony stimulating factor and a granulocyte macrophage colony stimulating factor, and optionally adding to the umbilical cord blood an effective amount of romiplostim, and optionally adding to the umbilical cord blood an effective amount of an erythropoiesis-stimulating agent, for forming a treated umbilical cord blood mixture for enhancing the engraftment of CD44 cells within said treated umbilical cord blood mixture. Preferably, the method of the present invention for enhancing umbilical cord blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the umbilical cord blood in any order of addition for forming a treated umbilical cord mixture of the umbilical cord blood, at least one of the granulocyte colony stimulating factor and the granulocyte macrophage colony stimulating factor, the romiplostim, and the erythropoiesis-stimulating agent. Another more preferred embodiment of the method of the present invention for enhancing umbilical cord blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the umbilical cord blood simultaneously for forming a treated umbilical cord mixture of the umbilical cord blood, at least one of the granulocyte colony stimulating factor and the granulocyte macrophage colony stimulating factor, the romiplostim, and the erythropoiesis-stimulating agent. In the methods of this invention, it is preferable that the erythropoiesis-stimulating agent is epoetin alfa. Another embodiment of these methods for enhancing umbilical cord blood engraftment include transplanting or transfusing the treated umbilical cord blood mixture to a patient.

In a most preferred embodiment of the present invention for enhancing umbilical cord blood engraftment, as described herein, includes wherein the amount of the granulocyte colony stimulating factor is at least about 10 ng/ml, wherein the erythropoiesis-stimulating agent is an epoetin alpha in an amount of at least about 5 Unit/ml, and the amount of the romiplostim is at least about 20 ng/ml.

Another embodiment of the present invention provides a method of enhancing a stem cell infusion comprising providing stem cells obtained from bone marrow, peripheral blood, or umbilical cord blood, adding to the stem cells an effective amount of at least one of a granulocyte colony stimulating factor and a granulocyte macrophage colony stimulating factor, and optionally adding to the stem cells an effective amount of romiplostim, and optionally adding to the stem cells an effective amount of an erythropoiesis-stimulating agent for forming a treated stem cell mixture for activating an up-regulation of a AF1q/CD44 signaling pathway. In a more preferred embodiment of this method of enhancing a stem cell infusion, as described herein, includes wherein (i) at least one of the granulocyte colony stimulating factor and the granulocyte macrophage colony stimulating factor, (ii) the romiplostim, and (iii) the erythropoiesis-stimulating agent, are added to the stem cells in any order of addition for forming a treated stem cell mixture of the stem cells, at least one of the granulocyte colony stimulating factor and the granulocyte macrophage colony stimulating factor, the romiplostim, and the erythropoiesis-stimulating agent. In another preferred embodiment of the method of enhancing a stem cell infusion, as described herein, includes wherein (i) at least one of the granulocyte colony stimulating factor and the granulocyte macrophage colony stimulating factor, (ii) the romiplostim, and (iii) the erythropoiesis-stimulating agent, are added to the stem cells simultaneously for forming a treated stem cell mixture of the stem cells, at least one of the granulocyte colony stimulating factor and the granulocyte macrophage colony stimulating factor, the romiplostim, and the erythropoiesis-stimulating agent. Preferably in these methods of this invention the erythropoiesis-stimulating agent is epoetin alfa. In a more preferred embodiment of the present method of enhancing a stem cell infusion, as described herein, the method includes wherein the amount of the granulocyte colony stimulating factor is at least about 10 ng/ml, the erythropoiesis-stimulating agent is epoetin alpha in an amount of at least about 5 Unit/ml, and the amount of the romiplostim is at least about 20 ng/ml. In yet another embodiment of the present invention of enhancing a stem cell infusion, as described herein, includes transplanting or infusing the treated stem cells to a patient.

Another embodiment of the present invention provides a method for enhancing bone marrow cells engraftment comprising providing a plurality of bone marrow cells, and adding to the bone marrow cells an effective amount of at least one of a granulocyte colony stimulating factor and a granulocyte macrophage colony stimulating factor, and optionally adding to the bone marrow cells an effective amount of romiplostim, and optionally adding to the bone marrow cells an effective amount of an erythropoiesis-stimulating agent for forming a treated bone marrow cell mixture for enhancing the engraftment of CD44 cells within the treated bone marrow cell mixture. In a preferred embodiment of the method for enhancing bone marrow cells engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the bone marrow cells in any order of addition for forming a treated bone marrow cell mixture of the bone marrow cells, at least one of the granulocyte colony stimulating factor and the granulocyte macrophage colony stimulating factor, the romiplostim, and the erythropoiesis-stimulating agent. In another preferred embodiment of this method for enhancing bone marrow cells engraftment, as described herein, the method includes wherein (i) at least one of the granulocyte colony stimulating factor and the granulocyte macrophage colony stimulating factor, (ii) the romiplostim, and (iii) the erythropoiesis-stimulating agent, are added to the bone marrow cells simultaneously for forming a treated bone marrow cell mixture of the bone marrow cells, at least one of the granulocyte colony stimulating factor and the granulocyte macrophage colony stimulating factor, the romiplostim, and the erythropoiesis-stimulating agent. In these methods of this invention, it is preferred that the erythropoiesis-stimulating agent is epoetin alfa. In a most preferred embodiment of this method for enhancing bone marrow cells engraftment, as described herein, includes wherein the amount of the granulocyte colony stimulating factor is at least about 10 ng/ml, the erythropoiesis-stimulating agent is an epoetin alpha in an amount of at least about 5 Unit/ml, and the amount of the romiplostim is at least about 20 ng/ml. In yet another embodiment of this method for enhancing bone marrow cells engraftment, as described herein, includes transplanting or transfusing the treated bone marrow cell mixture to a patient.

Another embodiment of the present invention includes a method for enhancing peripheral blood engraftment comprising providing an amount of a peripheral blood, adding to the peripheral blood an effective amount of at least one of a granulocyte colony stimulating factor and a granulocyte macrophage colony stimulating factor, and optionally adding to the peripheral blood an effective amount of romiplostim, and optionally adding to the peripheral blood an effective amount of an erythropoiesis-stimulating agent for forming a treated peripheral blood mixture for enhancing the engraftment of CD44 cells within the treated peripheral blood mixture. In a preferred embodiment of this method for enhancing peripheral blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the peripheral blood in any order of addition for forming a treated peripheral blood mixture of the peripheral blood, at least one of the granulocyte colony stimulating factor and the granulocyte macrophage colony stimulating factor, the romiplostim, and the erythropoiesis-stimulating agent. In a more preferred embodiment of this method for enhancing peripheral blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the peripheral blood simultaneously for forming a treated peripheral blood mixture of the peripheral blood, at least one of the granulocyte colony stimulating factor and the granulocyte macrophage colony stimulating factor, the romiplostim, and the erythropoiesis-stimulating agent. In the methods of this invention, it is preferable that the erythropoiesis-stimulating agent is epoetin alfa. In a most preferred method for enhancing peripheral blood engraftment, as described herein, the amount of the granulocyte colony stimulating factor is at least about 10 ng/ml, the erythropoiesis-stimulating agent is an epoetin alfa in an amount of at least about 5 Unit/ml, and the amount of the romiplostim is at least about 20 ng/ml. A further embodiment of the present method for enhancing peripheral blood engraftment, as described herein, includes transplanting or transfusing the treated peripheral blood mixture to a patient.

Another embodiment of this invention provides a method of enhancing a stem cell infusion comprising providing treated stem cells obtained from bone marrow, peripheral blood, or umbilical cord blood that are conditioned for activating an upregulation of a AF1q/CD44 signaling pathway for enhancing a stem cell infusion. The stem cells may be treated, such as for example but not limited to any of the methods of this invention as described herein, or variations thereof.

Another embodiment of this invention provides a method of activating an upregulation of a AF1q/CD44 signaling pathway comprising providing treated stem cells obtained from bone marrow, peripheral blood, or umbilical cord blood that are conditioned for activating an upregulation of a AF1q/CD44 signaling pathway.

Another embodiment of this invention provides for a stem cell product comprising treated stem cells obtained from bone marrow, peripheral blood, or umbilical cord blood that are conditioned for activating an upregulation of a AF1q/CD44 signaling pathway.

Another embodiment of this invention provides a stem cell product comprising a mixture of (i) stem cells obtained from bone marrow, peripheral blood, or umbilical cord blood, (ii) an effective amount of at least one of a granulocyte colony stimulating factor and a granulocyte macrophage colony stimulating factor, and (iii) optionally an effective amount of romiplostim, and (iv) optionally an effective amount of an erythropoiesis-stimulating agent, the mixture capable of activating an upregulation of a AF1q/CD44 signaling pathway.

Another embodiment of this invention provides a stem cell product comprising a mixture of (i) stem cells obtained from bone marrow, peripheral blood, or umbilical cord blood, (ii) an effective amount of at least one of a granulocyte colony stimulating factor and a granulocyte macrophage colony stimulating factor, and (iii) optionally an effective amount of romiplostim, and (iv) optionally an effective amount of an erythropoiesis-stimulating agent, the mixture capable of enhancing the engraftment of CD44 cells.

### BRIEF DESCRIPTION OF THE DRAWINGS

FIG. 1 is a Western blot analysis of the corresponding cell lysates showing AF1q expression was enhanced in the HMLE and MCF1Oa epithelial cells by using lenti-viral transduction technology.

FIG. 2 is a graph showing the expression of AF1q mRNA was enhanced by G-CSF, romiplostim (shown here as “NPLATE” or Nplate”, Amgen Inc.), an erythropoiesis-stimulating agent (shown here as “PROCRIT” or “Procrit”, Janssen Products, LP, an epoetin alfa), and a “MIX” (mixture of G-CSF, Nplate, and PROCRIT).

FIG. 3 is a graph showing the G-CSF enhanced chemotaxis of CD34+ cells.

FIG. 4 is a graph showing in vitro parameter (percent relative migration) to measure UCB homing/engraftment capacity of G-CSF, romiplostim (shown here as “NPLATE” or Nplate“, Amgen Inc.), an erythropoiesis-stimulating agent (shown here as “PROCRIT” or “Procrit”, Janssen Products, LP, an epoetin alfa), and a “All” (mixture of G-CSF, Nplate, and PROCRIT).

FIG. 5 is a graph showing concentrations of “Nplate” enhanced chemotaxis of peripheral blood CD34+ cells.

FIG. 6 is a graph showing concentration of “Procrit” enhanced chemotaxis of peripheral blood CD34+ cells.

### DETAILED DESCRIPTION OF THE INVENTION

The present invention provides a method for enhancing umbilical cord blood engraftment comprising providing an amount of an umbilical cord blood, adding to the umbilical cord blood an effective amount of at least one of a granulocyte colony stimulating factor and a granulocyte macrophage colony stimulating factor, and optionally adding to the umbilical cord blood an effective amount of romiplostim, and optionally adding to the umbilical cord blood an effective amount of an erythropoiesis-stimulating agent, for forming a treated umbilical cord blood mixture for enhancing the engraftment of CD44 cells within said treated umbilical cord blood mixture. Preferably, the method of the present invention for enhancing umbilical cord blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the umbilical cord blood in any order of addition for forming a treated umbilical cord mixture of the umbilical cord blood, at least one of the granulocyte colony stimulating factor and the granulocyte macrophage colony stimulating factor, the romiplostim, and the erythropoiesis-stimulating agent. Another more preferred embodiment of the method of the present invention for enhancing umbilical cord blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the umbilical cord blood simultaneously for forming a treated umbilical cord mixture of the umbilical cord blood, at least one of the granulocyte colony stimulating factor and the granulocyte macrophage colony stimulating factor, the romiplostim, and the erythropoiesis-stimulating agent. Preferably, in these methods of this invention, the erythropoiesis-stimulating agent is epoetin alfa. Another embodiment of these methods for enhancing umbilical cord blood engraftment include transplanting or transfusing the treated umbilical cord blood mixture to a patient.

As used herein, the term erythropoiesis-stimulating agent (commonly known in the art as an “ESA”) includes, such as for example, but not limited to epoetin alfa (commercially available as EPOGEN, a registered trademark of Amgen Inc., and PROCRIT, a registered trademark of Janssen Products, LP), and darbepoetin alfa (commercially available as ARANSEP, a registered trademark of Amgen Inc.). Broadly, the term erythropoiesis-stimulating agent, as used herein encompasses erythropoietin, a glycoprotein hormone that stimulates the production of red blood cells by bone marrow.

In a most preferred embodiment of the present invention for enhancing umbilical cord blood engraftment, as described herein, includes wherein the amount of the granulocyte colony stimulating factor is at least about 10 ng/ml, wherein the erythropoiesis-stimulating agent is an epoetin alfa in an amount of at least about 5 Unit/ml, and the amount of the romiplostim is at least about 20 ng/ml.

Another embodiment of the present invention provides a method of enhancing a stem cell infusion comprising providing stem cells obtained from bone marrow, peripheral blood, or umbilical cord blood, adding to the stem cells an effective amount of at least one of a granulocyte colony stimulating factor and a granulocyte macrophage colony stimulating factor, and optionally adding to the stem cells an effective amount of romiplostim, and optionally adding to the stem cells an effective amount of an erythropoiesis-stimulating agent for forming a treated stem cell mixture for activating an up-regulation of a AF1q/CD44 signaling pathway. In a more preferred embodiment of this method of enhancing a stem cell infusion, as described herein, includes wherein (i) at least one of the granulocyte colony stimulating factor and the granulocyte macrophage colony stimulating factor, (ii) the romiplostim, and (iii) the erythropoiesis-stimulating agent are added to the stem cells in any order of addition for forming a treated stem cell mixture of the stem cells, at least one of the granulocyte colony stimulating factor and the granulocyte macrophage colony stimulating factor, the romiplostim, and the erythropoiesis-stimulating agent. In another preferred embodiment of the method of enhancing a stem cell infusion, as described herein, includes wherein (i) at least one of the granulocyte colony stimulating factor and the granulocyte macrophage colony stimulating factor, (ii) the romiplostim, and (iii) the erythropoiesis-stimulating agent, are added to the stem cells simultaneously for forming a treated stem cell mixture of the stem cells, at least one of the granulocyte colony stimulating factor and the granulocyte macrophage colony stimulating factor, the romiplostim, and the erythropoiesis-stimulating agent. Preferably, in these methods of this invention, the erythropoiesis-stimulating agent is epoetin alfa. In a more preferred embodiment of the present method of enhancing a stem cell infusion, as described herein, the method includes wherein the amount of the granulocyte colony stimulating factor is at least about 10 ng/ml, wherein the erythropoiesis-stimulating agent is an epoetin alfa in an amount of at least about 5 Unit/ml, and the mount of the romiplostim is at least about 20 ng/ml. In yet another embodiment of the present invention of enhancing a stem cell infusion, as described herein, includes transplanting or infusing the treated stem cells to a patient.

Another embodiment of the present invention provides a method for enhancing bone marrow cells engraftment comprising providing a plurality of bone marrow cells, and adding to the bone marrow cells an effective amount of at least one of a granulocyte colony stimulating factor and a granulocyte macrophage colony stimulating factor, and optionally adding to the bone marrow cells an effective amount of romiplostim, and optionally adding to the bone marrow cells an effective amount of an erythropoiesis-stimulating agent for forming a treated bone marrow cell mixture for enhancing the engraftment of CD44 cells within the treated bone marrow cell mixture. In a preferred embodiment of the method for enhancing bone marrow cells engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the bone marrow cells in any order of addition for forming a treated bone marrow cell mixture of the bone marrow cells, at least one of the granulocyte colony stimulating factor and the granulocyte macrophage colony stimulating factor, the romiplostim, and the erythropoiesis-stimulating agent. In another preferred embodiment of this method for enhancing bone marrow cells engraftment, as described herein, the method includes wherein (i) at least one of the granulocyte colony stimulating factor and the granulocyte macrophage colony stimulating factor, (ii) the romiplostim, and (iii) the erythropoiesis-stimulating agent, are added to the bone marrow cells simultaneously for forming a treated bone marrow cell mixture of the bone marrow cells, at least one of the granulocyte colony stimulating factor and the granulocyte macrophage colony stimulating factor, the romiplostim, and the erythropoiesis-stimulating agent. Preferably, in these methods of this invention, the erythropoiesis-stimulating agent is epoetin alfa. In a most preferred embodiment of this method for enhancing bone marrow cells engraftment, as described herein, includes wherein the amount of the granulocyte colony stimulating factor is at least about 10 ng/ml, wherein the erythropoiesis-stimulating agent is an epoetin alfa in an amount of at least about 5 Unit/ml, and the amount of the romiplostim is at least about 20 ng/ml. In yet another embodiment of this method for enhancing bone marrow cells engraftment, as described herein, includes transplanting or transfusing the treated bone marrow cell mixture to a patient.

Another embodiment of the present invention includes a method for enhancing peripheral blood engraftment comprising providing an amount of a peripheral blood, adding to the peripheral blood an effective amount of at least one of a granulocyte colony stimulating factor and a granulocyte macrophage colony stimulating factor, and optionally adding to the peripheral blood an effective amount of romiplostim, and optionally adding to the peripheral blood an effective amount of an erythropoiesis-stimulating agent for forming a treated peripheral blood mixture for enhancing the engraftment of CD44 cells within the treated peripheral blood mixture. In a preferred embodiment of this method for enhancing peripheral blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the peripheral blood in any order of addition for forming a treated peripheral blood mixture of the peripheral blood, at least one of the granulocyte colony stimulating factor and the granulocyte macrophage colony stimulating factor, the romiplostim, and the erythropoiesis-stimulating agent. In a more preferred embodiment of this method for enhancing peripheral blood engraftment, as described herein, includes wherein (i) at least one of the granulocyte colony stimulating factor and the granulocyte macrophage colony stimulating factor, (ii) the romiplostim, and (iii) the erythropoiesis-stimulating agent, are added to the peripheral blood simultaneously for forming a treated peripheral blood mixture of the peripheral blood, at least one of the granulocyte colony stimulating factor and the granulocyte macrophage colony stimulating factor, the romiplostim, and the erythropoiesis-stimulating agent. Preferably, in these methods of this invention, the erythropoiesis-stimulating agent is epoetin alfa. In a most preferred method for enhancing peripheral blood engraftment, as described herein, the amount of the granulocyte colony stimulating factor is at least about 10 ng/ml, wherein the erythropoiesis-stimulating agent is an epoetin alfa in an amount of at least about 5 Unit/ml, and the amount of the romiplostim is at least about 20 ng/ml. A further embodiment of the present method for enhancing peripheral blood engraftment, as described herein, includes transplanting or transfusing the treated peripheral blood mixture to a patient.

Another embodiment of this invention provides a method of enhancing a stem cell infusion comprising providing treated stem cells obtained from bone marrow, peripheral blood, or umbilical cord blood that are conditioned for activating an upregulation of a AF1q/CD44 signaling pathway for enhancing a stem cell infusion. The stem cells may be treated, such as for example but not limited to any of the methods of this invention as described herein, or variations thereof.

Another embodiment of this invention provides a method of activating an upregulation of a AF1q/CD44 signaling pathway comprisingproviding treated stem cells obtained from bone marrow, peripheral blood, or umbilical cord blood that are conditioned for activating an upregulation of a AF1q/CD44 signaling pathway. The stem cells may be treated, such as for example but not limited to any of the methods of this invention as described herein, or variations thereof.

Another embodiment of this invention provides for a stem cell product comprising treated stem cells obtained from bone marrow, peripheral blood, or umbilical cord blood that are conditioned for activating an upregulation of a AF1q/CD44 signaling pathway. The stem cells may be treated, such as for example but not limited to any of the methods of this invention as described herein, or variations thereof.

Another embodiment of this invention provides a stem cell product comprising a mixture of (i) stem cells obtained from bone marrow, peripheral blood, or umbilical cord blood, (ii) an effective amount of at least one of a granulocyte colony stimulating factor and a granulocyte macrophage colony stimulating factor, and (iii) optionally an effective amount of romiplostim, and (iv) optionally an effective amount of an erythropoiesis-stimulating agent, the mixture capable of activating an upregulation of a AF1q/CD44 signaling pathway.

Another embodiment of this invention provides a stem cell product comprising a mixture of (i) stem cells obtained from bone marrow, peripheral blood, or umbilical cord blood, (ii) an effective amount of at least one of a granulocyte colony stimulating factor and a granulocyte macrophage colony stimulating factor, and (iii) optionally an effective amount of romiplostim, and (iv) optionally an effective amount of an erythropoiesis-stimulating agent, the mixture capable of enhancing the engraftment of CD44 cells.

Allogeneic stem cell transplantation is an effective approach to cure many hematopoietic malignant disorders. The chance of getting a minimal matched UCB for transplantation in a given recipient is more than 95%. Umbilical cord blood (UCB) is a valuable alternative source of hematopoietic stem cells for patients that require allogeneic transplantation in the absence of readily available human leukocyte antigen (HLA) matched marrow and blood hematopoietic stem cells.

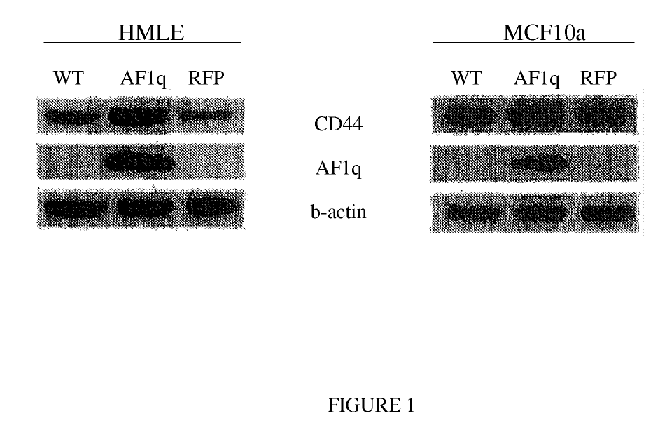

The AF1q gene is a mixed lineage leukemia (MLL) fusion partner in acute myeloid leukemia (AML) patients whose leukemic blasts carrying a t(1; 11) (q21; q23) translocation. AF1q is expressed in normal hematopoietic precursors including CD34+ cells, but has very little or no expression in mature hematopoietic blood cells. In the present invention, the inventor has found that activated AF1q can enhance hematopoietic cells and epithelial cell trans-well migration through direct transcriptional activation of CD44 which is known as an important hematopoietic cell engraftment molecule (see FIG. 1). FIG. 1 shows that CD44 can be activated through enhancing the AF1q expression. AF1q expression was enhanced in the HMLE and MCF10a epithelial cells by using lenti-viral transduction technology (known by those skilled in the art). CD44 protein production was significantly increased in the AF1q panels compared with wild-type and control panels in both HMLE and MCF1Oa cells by Western blot analysis of the corresponding cell lysates.

CD44 plays important roles in hematopoietic cell homing/migration due to its essential role in cell-to-extracellular matrix adhesion. Activating the AF1q/CD44 signaling significantly enhances the UCB homing/engraftment manifested by promotion of the UCB trans-well migration (an in vitro parameter to measure the UCB homing/engraftment capacity) by transcriptional activation of the AF1q/CD44 signaling pathway. For successful bone marrow transplantation, hematopoietic stem cells (HSCs) must pass along the peripheral blood and overpass through endothelial cells of the bone marrow. The HSCs must also migrate and be home to a supportive microenvironment. In this respect, increased CD44 activity can potentially be one of a vital constituent that improves the safety of the UCB transplantation for adult recipients.

AF1q expression in hematopoietic tissues responds to growth factors stimulation, such as G-CSF (granulocyte-colony stimulating factors). G-CSF is routinely given to the adult and pediatric UCB recipients at a daily base within the first week of UCB transplantation until the evidence of neutrophil engraftment is seen which is when the absolute neutrophil number >1000-1500. The present invention's ex-vivo studies have confirmed that UCB exposed to at least one of a G-CSF and GM-CSF (granulocyte/macrophage colony-stimulating factors), or erythropoietin, or an erythropoiesis-stimulating agent, or romiplostim, or combinations thereof, for an effective time as short as 15 minutes to about one hour, activates the AF1q/CD44 signaling pathway resulting in a significantly enhancement of UCB trans-well migration (in-vitro parameter to measure UCB homing/engraftment capacity). The effective time for AF1q/CD44 activation efficiency as short as 15 minutes is based on the minimal time requirement from thawing the UCB from storage to bedside infusion at the transplantation ward. The biologically desirable UCB unit exposed to one of the G-CSF and GM-CSF growth factors enhances this UCB unit's engraftment capacity over its competitive unit (no growth factors exposure).

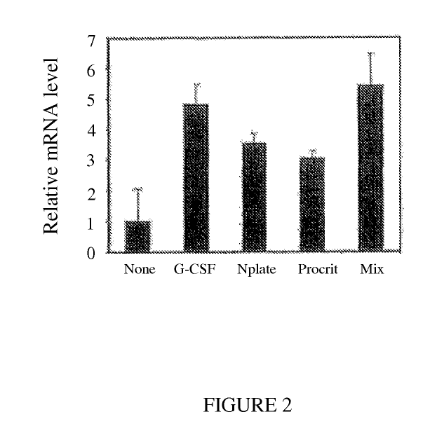

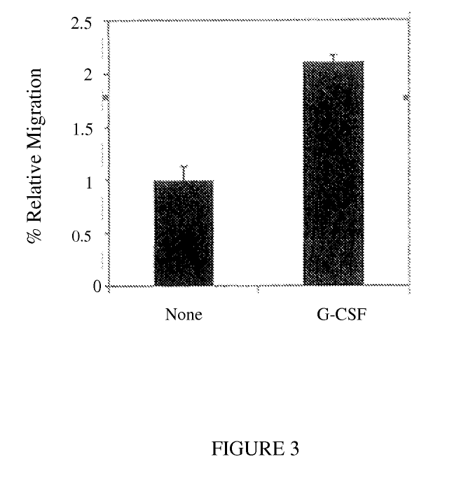

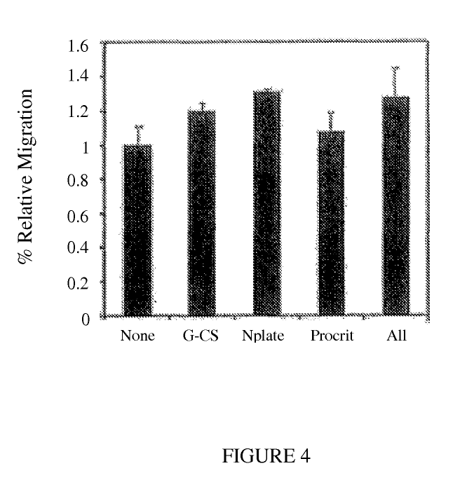

FIG. 2 shows in vitro parameter to measure UCB homing/engraftment capacity. FIG. 2 data was produced by a real-time PCR technique to determine the AF1q mRNA production as an indirect parameter of AF1q protein production. This was performed because the CD34 cell number in each UCB unit is insufficient to perform a Western blot. The observations are reliable because the peripheral blood CD34 cells are confirmed with elevated AF1q mRNA is corresponding to the elevated AF1q protein production. FIG. 2 shows that the sharp increase in the AF1q mRNA production is in response to exposure of G-CSF, erythropoiesis-stimulating agent, romplostin, and their combination (mix of G-CSF, erythropoiesis-stimulating agent, and romiplostim) in the UCB CD34 population. Those skilled in the art understand that that G-CSF is available from several sources, such as for example, filgrastim, a human granulocyte colony-stimulating factor produced by recombinant DNA technology and commercially available as NEUPOGEN, a registered trademark of Amgen Inc.; that GM-CSF is available from several sources, such as for example, recombinant human GM-CSF commercially available from Peprotech; that erythropoiesis-stimulating agent is commercially available as PROCRIT, a registered trademark of Janssen Products, LP.; and that romiplostim is commercially available as NPLATE, a registered trademark of Amgen, Inc. FIG. 2 shows that while AF1q expression levels are increased by the individual drugs alone, the combination of G-CSF, erythropoiesis-stimulating agent, and romiplostim significantly increases the AF1q expression level beyond use of any of the drugs alone. Targets were normalized to reactions performed by using B2M (p<0.05). These observations are especially important in clinical practice that G-CSF or erythropoietin or their combination is used routinely for hematopoietic stem cell recipients when they are in the risk of primary or secondary graft failure of any reason. It has also been confirmed that UCB CD34 cells gained strong trans-well migration ability after shortly exposure to the individual growth factors (G-CSF) and its combination with erythropoiesis-stimulating agent and romiplostim. Cell migration in response to stimuli is central to a broad range of physiological processes, including immune responses, wound healing, and stem cell homing. The UCB CD34 cells averagely gain 30-50% higher of trans-well migration capacity compared to control as shown in FIGS. 3 and 4.

FIG. 3 shows the G-CSF enhanced chemotaxis of CD34+ cells. FIG. 3 shows the results of chemotactic responses of CD34+ cells that were assayed in a 24-well chemotaxis chamber. The stroma cells were plated in the lower wells of the chamber before one day, and SDF-1 condition medium (stromal cell-derived factor-1 commercially available from several sources, such as Research & Diagnostics Systems, Inc.) (100 ng/ml) was added in the lower wells of the chamber the very day of experiment. The CD34+ (1.5×10⁵) cells were treated with G-CSF for 1 hour and added to the upper wells. Non-treated CD34+ cells were used as a control. The upper and lower wells were separated by a 5-μm pore size polycarbonate filter. The chamber was incubated for 4 hours at 37° C. (Centigrade), and the cells migrating to the bottom wells were counted by FACS Calibur. Values were compared with the ability of CD+34 cells upon no treatment. P<0.05.

FIG. 4 shows chemotactic migration ability of CD34+ cells by stimulation of G-CSF (10 ng/ml), NPLATE (Amgen, Inc) (20 ng/ml), and PROCRIT (Janssen Products, LP) (5 Unit/ml) each alone, and as a combination of a mixture of G-CSF (10 ng/ml), NPLATE (Amgen, Inc) (20 ng/ml), and PROCRIT (Janssen Products, LP) (5 Unit/ml) shown in FIG. 4 as “All” on the x-axis of the graph. Chemotactic responses of CD34+ cells were assayed in a 24-well chemotaxis chamber. Condition medium from stromal cells was used as a chemo-attractant. The CD34+ (1×10⁵) cells were treated with each of G-CSF, NPLATE, and PROCRIT, alone, and as a mixture “All” for 1 hour and added to the upper wells. The upper and lower wells were separated by a 5 μm pore size polycarbonate filter. The chamber was incubated for 4 hours at 37° C. (37 degrees centigrade) and the cells migrating to the bottom wells were counted by FACS Calibur. Values were compared with the ability of CD34+ cells upon no treatment drug P<0.05. Therefore, those skilled in the art will understand that the present invention, in a preferred embodiment, discloses a method of treating UCB with a combination of G-CSF, NPLATE, and PROCRIT with optimal concentration as set forth herein is a valuable process to achieve outstanding migration results and for increasing the ability to induce AF1q expression and enhance the homing of UCB for use in transplantation.

In a preferred embodiment of this invention, a method is provided comprising adding an amount of G-CSF, erythropoiesis-stimulating agent, and romiplostim simultaneously for inducing AF1q expression and enhancing homing of bone marrow cells for use in transplantation. As a result, treated bone marrow cells acquire more ability of homing. In a more preferred embodiment, this method, as described herein, includes wherein the treated bone marrow cells (1×10⁶) are transplanted after treatment with the drugs shown in Table 1 below. Most preferably, the method includes treating the cells with said drugs from about 15 minutes to at least one 1 hour.

TABLE 1
The drugs concentration for treatment.

| Drugs | Concentration |
|---|---|
| G-CSF | 10 ng/ml |
| NPLATE (Nplate) | 20 ng/ml |
| PROCRIT | 5 Unit/ml |

It will be appreciated by those persons skilled in the art that stem cell transplantation is to cure many different cancers and diseases. Stem cells for transplantation can be collected from bone marrow, peripheral blood and umbilical cord blood. The type of transplant will depend on where the cells are from. According to the source of the cells, it is known as Autologous transplant, Allogeneic related transplant, Allogeneic unrelated transplant and Syngeneic transplant.

It has now been found that AF1q is major role in cell migration. It is therefore an object of the present invention to provide the use of G-CSF, an erythropoiesis-stimulating agent (preferably NPLATE, also referred to as “Nplate” herein), and PROCRIT for enhancing homing and adaption of transplanted cells as a result.

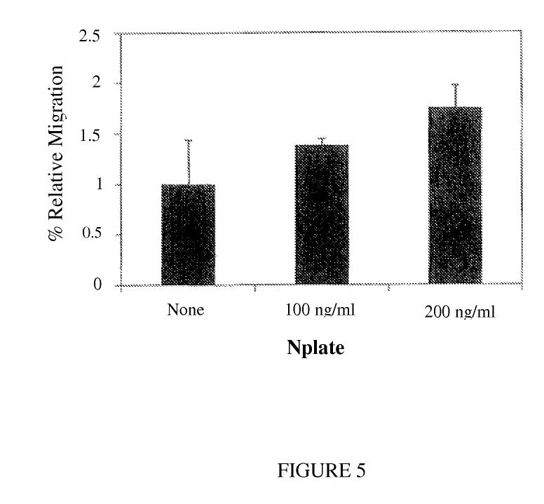

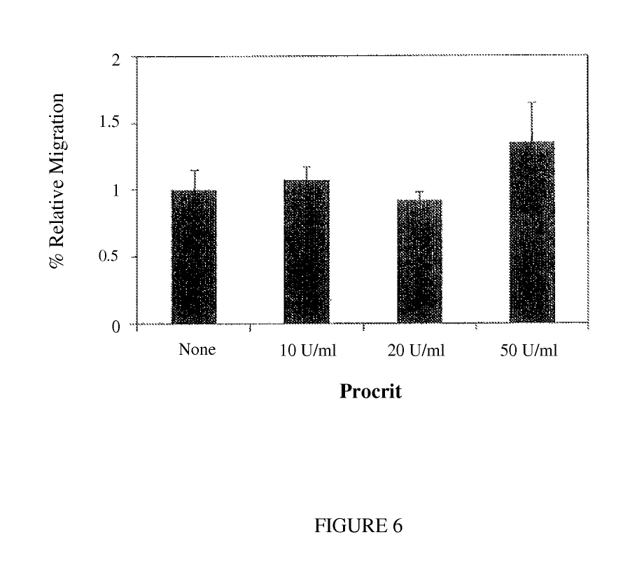

Thus, it will be appreciated by those persons skilled in the art that the present invention provides a method comprising pretreating a unit of UCB cells with one or more of G-CSF, erythropoietin, erythropoiesis-stimulating agent, and romplostin, and preferably a combination of (i) one or more of a G-CSF and GM-CSF, (ii) erythropoiesis-stimulating agent, and (iii) romiplostim, increases the ability to induce AF1q expression and enhance the homing of treated stem cells (whether the cells are from UCB, peripheral blood, or bone marrow) for use in transplantation. As a result, for example but not limited to, treated bone marrow cells acquire more ability of homing and increased cellular migration. When the stem cells are separately treated with at least 10 ng of G-CSF, 5 units of epoetin alfa, and 20 ng of romiplostim, the treatment induces AF1q expression and enhances the homing of an effective amount of bone marrow cells for use in transplantation, however, the establishment of the preferred embodiment of this invention is the ratio of at least 10 ng of G-CSF, 5 units of epoetin alfa, and 20 ng of romiplostim to achieve the best results. FIGS. 5 and 6 show that NPLATE and PROCRIT slightly enhanced chemotaxis of CD34+ cells from peripheral blood. These cells were treated with G-CSF during collecting. This data indicate that the methods of the present invention can also be used in marrow and peripheral blood stem cells transplantation as well. Stem cells for transplantation can be collected from bone marrow, peripheral blood and cord blood.

The treatment of the stem cells with monoclonal antibody H90, which activates CD44, induces the differentiation of AML cells in vivo and alters the stem cell properties of AML LSCs which is not preferable for use with the method of this invention. In addition, H90 treatment reduced the ability of AML LSCs to home to the bone marrow and spleen. This could be significant as the HSCs treated with this process highly express CD44. Therefore, the method of the present invention should not be simultaneously used with any reagents which are able to activate or bind to CD44.

The present invention provides for the activation of the AF1q/CD44 signaling by hematopoietic growth factors before stem cell infusion, such as marrow, peripheral, and UCB can enhance engraftment and promote engraftment capacity. Selectively activating AF1q/CD44 signaling in a before stem cell infusion unit over the competing unit will allow the activated unit to have an engraftment advantage compared to the other. AF1q/CD44 can be activated by one or more of G-CSF, GM-CSF, erythropoietin, erythropoiesis-stimulating agent, and romiplostim, and combinations thereof. The activation is not dose responsive and appears to be all or none in effect.

These terms and specifications, including the examples, serve to describe the invention by example and not to limit the invention. Whereas particular embodiments of this invention have been described for purposes of illustration, it will be evident to those persons skilled in the art that numerous variations of the details of the present invention may be made without departing from the invention as defined herein and in the appended claims.

What is claimed is:

1. A method for enhancing umbilical cord blood engraftment comprising:
providing an amount of an umbilical cord blood;
adding to said umbilical cord blood an effective amount of at least one of a granulocyte colony stimulating factor and a granulocyte macrophage colony stimulating factor;
optionally adding to said umbilical cord blood an effective amount of romiplostim; and
optionally adding to said umbilical cord blood an effective amount of an erythropoiesis-stimulating agent for forming a treated umbilical cord blood mixture for enhancing the engraftment of CD44 cells within said treated umbilical cord blood mixture.

2. The method of claim 1 including wherein (i) at least one of said granulocyte colony stimulating factor and said granulocyte macrophage colony stimulating factor, (ii) said romiplostim, and (iii) said erythropoiesis-stimulating agent, are added to said umbilical cord blood in any order of addition for forming said treated umbilical cord mixture of said umbilical cord blood, at least one of said granulocyte colony stimulating factor and a granulocyte macrophage colony stimulating factor, said romiplostim, and said erythropoiesis-stimulating agent.

3. The method of claim 1 including wherein (i) at least one of said granulocyte colony stimulating factor and said granulocyte macrophage colony stimulating factor, (ii) said romiplostim, and (iii) said erythropoiesis-stimulating agent, are added to said umbilical cord blood simultaneously for forming said treated umbilical cord mixture of said umbilical cord blood, at least one of said granulocyte colony stimulating factor and a granulocyte macrophage colony stimulating factor, said romiplostim, and said erythropoiesis-stimulating agent.

4. The method of claim 1 including transplanting or transfusing said treated umbilical cord blood mixture to a patient.

5. The method of claim 1 including wherein said amount of said granulocyte colony stimulating factor is at least about 10 ng/ml, wherein said erythropoiesis-stimulating agent is epoetin alfa in an amount of at least about 5 Unit/ml, and said amount of said romiplostim is at least about 20 ng/ml.

6. A method of enhancing a stem cell infusion comprising:
providing stem cells obtained from bone marrow, peripheral blood, or umbilical cord blood;
adding to said stem cells an effective amount of at least one of a granulocyte colony stimulating factor and a granulocyte macrophage colony stimulating factor;
optionally adding to said stem cells an effective amount of romiplostim; and
optionally adding to said stem cells an effective amount of an erythropoiesis-stimulating agent for forming a treated stem cell mixture for activating an upregulation of a AF1q/CD44 signaling pathway.

7. The method of claim 6 including wherein (i) at least one of said granulocyte colony stimulating factor and said granulocyte macrophage colony stimulating factor, (ii) said romiplostim, and (iii) said erythropoiesis-stimulating agent, are added to said stem cells in any order of addition for forming a treated stem cell mixture of said stem cells, at least one of said granulocyte colony stimulating factor and a granulocyte macrophage colony stimulating factor, said romiplostim, and said erythropoiesis-stimulating agent.

8. The method of claim 6 including wherein (i) at least one of said granulocyte colony stimulating factor and said granulocyte macrophage colony stimulating factor, (ii) said romiplostim, and (iii) said erythropoiesis-stimulating agent, are added to said stem cells simultaneously for forming a treated stem cell mixture of said stem cells, at least one of said granulocyte colony stimulating factor and a granulocyte macrophage colony stimulating factor, said romiplostim, and said erythropoiesis-stimulating agent.

9. The method of claim 6 wherein said amount of said granulocyte colony stimulating factor is at least about 10 ng/ml, wherein said erythropoiesis-stimulating agent is an epoetin alfa in an amount of at least about 5 Unit/ml, and said amount of said romiplostim is at least about 20 ng/ml.

10. The method of claim 6 including transplanting or infusing said treated stem cells to a patient.

11. A method for enhancing bone marrow cells engraftment comprising:
providing a plurality of bone marrow cells;
adding to said bone marrow cells an effective amount of at least one of a granulocyte colony stimulating factor and a granulocyte macrophage colony stimulating factor;
optionally adding to said bone marrow cells an effective amount of romiplostim; and
optionally adding to said bone marrow cells an effective amount of erythropoiesis-stimulating agent for forming a treated bone marrow cell mixture for enhancing the engraftment of CD44 cells within said treated bone marrow cell mixture.

12. The method of claim 11 including wherein (i) at least one of said granulocyte colony stimulating factor and said granulocyte macrophage colony stimulating factor, (ii) said romiplostim, and (iii) said erythropoiesis-stimulating agent, are added to said bone marrow cells in any order of addition for forming a treated bone marrow cell mixture of said bone marrow cells, at least one of said granulocyte colony stimulating factor and a granulocyte macrophage colony stimulating factor, said romiplostim, and said erythropoiesis-stimulating agent.

13. The method of claim 11 including wherein (i) at least one of said granulocyte colony stimulating factor and said granulocyte macrophage colony stimulating factor, (ii) said romiplostim, and (iii) said erythropoiesis-stimulating agent, are added to said bone marrow cells simultaneously for forming a treated bone marrow cell mixture of said bone marrow cells, at least one of said granulocyte colony stimulating factor and a granulocyte macrophage colony stimulating factor, said romiplostim, and said erythropoiesis-stimulating agent.

14. The method of claim 11 wherein said amount of said granulocyte colony stimulating factor is at least about 10 ng/ml, wherein said erythropoiesis-stimulating agent is an epoetin alfa in an amount of at least about 5 Unit/ml, and said amount of said romiplostim is at least about 20 ng/ml.

15. The method of claim 11 including transplanting or transfusing said treated bone marrow cell mixture to a patient.

16. A method for enhancing peripheral blood engraftment comprising:
providing an amount of a peripheral blood;
adding to said peripheral blood an effective amount of at least one of a granulocyte colony stimulating factor and a granulocyte macrophage colony stimulating factor;
optionally adding to said peripheral blood an effective amount of romiplostim; and
optionally adding to said peripheral blood an effective amount of an erythropoiesis-stimulating agent for forming a treated peripheral blood mixture for enhancing the engraftment of CD44 cells within said treated peripheral blood mixture.

17. The method of claim 16 including wherein (i) at least one of said granulocyte colony stimulating factor and said granulocyte macrophage colony stimulating factor, (ii) said romiplostim, and (iii) said erythropoiesis-stimulating agent, are added to said peripheral blood in any order of addition for forming a treated peripheral blood mixture of said peripheral blood, at least one of said granulocyte colony stimulating factor and a granulocyte macrophage colony stimulating factor, said romiplostim, and said erythropoiesis-stimulating agent.

18. The method of claim 16 including wherein (i) at least one of said granulocyte colony stimulating factor and said granulocyte macrophage colony stimulating factor, (ii) said romiplostim, and (iii) said erythropoiesis-stimulating agent, are added to said peripheral blood simultaneously for forming a treated peripheral blood mixture of said peripheral blood, at least one of said granulocyte colony stimulating factor and a granulocyte macrophage colony stimulating factor, said romiplostim, and said erythropoiesis-stimulating agent.

19. The method of claim 16 wherein said amount of said granulocyte colony stimulating factor is at least about 10 ng/ml, wherein said erythropoiesis-stimulating agent is an epoetin alfa in an amount of at least about 5 Unit/ml, and said amount of said romiplostim is at least about 20 ng/ml.

20. The method of claim 16 including transplanting or transfusing said treated peripheral blood mixture to a patient.

21. A method of enhancing a stem cell infusion comprising:
providing treated stem cells obtained from bone marrow, peripheral blood, or umbilical cord blood that are conditioned for activating an upregulation of a AF1q/CD44 signaling pathway for enhancing a stem cell infusion.

22. A method of activating an upregulation of a AF1q/CD44 signaling pathway comprising:
providing treated stem cells obtained from bone marrow, peripheral blood, or umbilical cord blood that are conditioned for activating an upregulation of a AF1q/CD44 signaling pathway.

23. A stem cell product comprising:
treated stem cells obtained from bone marrow, peripheral blood, or umbilical cord blood that are conditioned for activating an upregulation of a AF1q/CD44 signaling pathway

24. A stem cell product comprising:
a mixture of (i) stem cells obtained from bone marrow, peripheral blood, or umbilical cord blood, (ii) an effective amount of at least one of a granulocyte colony stimulating factor and a granulocyte macrophage colony stimulating factor, and (iii) optionally an effective amount of romiplostim, and (iv) optionally an effective amount of an erythropoiesis-stimulating agent, said mixture capable of activating an upregulation of a AF1q/CD44 signaling pathway.

25. A stem cell product comprising:
a mixture of (i) stem cells obtained from bone marrow, peripheral blood, or umbilical cord blood, (ii) an effective amount of at least one of a granulocyte colony stimulating factor and a granulocyte macrophage colony stimulating factor, and (iii) optionally an effective amount of romiplostim, and (iv) optionally an effective amount of an erythropoiesis-stimulating agent, said mixture capable of enhancing the engraftment of CD44 cells.
